(19) United States
(12) Patent Application Publication
(10) Pub. No.: US 2025/0010035 A1
(43) Pub. Date: Jan. 9, 2025

(54) STEERABLE DEVICE AND SYSTEM

(71) Applicant: ECOLE POLYTECHNIQUE FEDERALE DE LAUSANNE (EPFL), Lausanne (CH)

(72) Inventors: Guillaume PETITPIERRE, Veyrier (CH); Marc BOERS, Kerns (CH); Philippe RENAUD, Preverenges (CH)

(21) Appl. No.: 18/741,231

(22) Filed: Jun. 12, 2024

Related U.S. Application Data
(62) Division of application No. 16/757,632, filed on Apr. 20, 2020, now abandoned.

(51) Int. Cl. A61M 25/01; A61M 25/09

(52) U.S. Cl. CPC A61M 25/0138; A61M 25/0136; A61M 25/0147; A61M 25/09033

(57) ABSTRACT

The disclosure concerns a steerable device for use as e.g. a guidewire for insertion into a subject's body. The device features a single side deflection of a bendable portion located at its distal end by application of a longitudinally-directed force either on an inner pull wire or on the bendable portion. The bendable portion is characterized by the presence of a reinforcement structure on one lateral side and a stress relief portion on the opposed lateral side that, upon actuation of the device, allows single side bending thanks to the physical constriction of the reinforcement structure, limiting the compression of one side of the bendable portion. The disclosure further concerns a system further comprising the steerable device and an actuator, as well as methods for using thereof.

This application is a divisional application of U.S. patent application Ser. No. 16/757,632 filed Apr. 20, 2020, which is the U.S. national phase of International Application No. PCT/IB2017/056592 filed 24 Oct. 2017, which designated the U.S., the entire contents of each of which are hereby incorporated by reference.

### TECHNICAL FIELD

The present invention belongs to the field of microtechnology. In particular, the invention features a steerable device and system useful for instance as a guidewire or catheter for surgical operations.

### BACKGROUND ART

Interventional radiology (IR) is a sub-speciality of radiology using minimally invasive image-guided procedures to diagnose and treat nearly all organs of the human body. The medical procedure mainly relies on both catheters allowing access through the vascular system (also includes the biliary tract, the gastrointestinal tract, etc.) and imaging methods (fluoroscopy, ultrasound, computed tomography) which allows precise navigation. IR is largely minimally invasive, as it takes advantage of the naturally present blood distribution system; for instance, vascular access is often provided through a single femoral artery/vein entry point, thus minimizing the risk to the patients and improving health outcomes. These procedures have been demonstrated to be less risky, to produce less pain and to decrease the recovery time in comparison to open surgeries.

Medical devices in IR are mainly composed of guidewires and catheters. At least the tip of these elements are radiopaque to enable image-guided navigation. The guidewires are introduced first in the vascular system and advanced in the blood vessels until reaching the targeted location. Catheters, which comprise a lumen, are advanced over guidewires which serve as a guiding element to reach the targeted location as well. Once the catheter is in place, it is used as a support channel for the introduction of other specific catheter devices aiming, for instance, at revascularization or embolization.

Guidewires have therefore the critical role of providing the initial access in order to reach a targeted region. Current guidewires can be either translated and/or rotated during this operation. They most often comprise a bent-shaped tip by which, through the external rotation and translation of the guidewire, the surgeon can select vascular intersections. Combined with imaging methods, this allows the surgeon to navigate the guidewire to the intended region.

The shape of the guidewire distal tip therefore largely impacts the navigation success of the device through the vascular system. Currently, guidewires are proposed with different pre-bent shapes, the surgeon selecting the best suited option according to the vessels geometry and the region he wants to reach. During the procedure, he commonly needs to replace the guidewire by another, more appropriate one. Similarly, the guidewire is often extracted from the patient, bent by hand by the surgeon (some guidewires propose this feature), before being re-introduced in the patient. This results in increased surgery time and procedure-related risks such as infections.

Guidewires replacement and distal tip repeated bending (by hand) is particularly frequent when the surgeon is confronted with tortuous blood vessels. This includes (and is not limited to) the neurovascular system, the cardiovascular system and the peripheral vascular system. In general, the blood vessels are more prone to present irregular, tortuous shapes at the distal portions of the vascular system (e.g. second and third level of the brain arteries).

Several solutions have been proposed in the past for solving the above shortcomings, in particular by providing steerable surgical tools to facilitate the surgeons' operation. For instance, US Patent Application 2016/0250449 describes a neuro-surgery assembly including a neuro-catheter slidably received over a wire guide. The wire guide includes an inner tube positioned within an elongated hollow tube that includes a pattern of openings at its distal segment to provide an atraumatic tip for reaching sensitive locations in the brain. The wire guide is changeable between a stiff condition in which the inner tube is pressurized and a soft condition in which the inner tube is depressurized. The user may switch between the stiff and soft conditions to negotiate the tortuous pathway to a brain treatment site without a need to swap out to a different wire guide to support advancement of the neuro-catheter.

WO 2015/164912 discloses an elongate, steerable apparatus such as a guidewire including an elongated body having at least one inner lumen, one, or more preferably multiple, tendons coupled to a distal bending region (e.g., the distal tip region) at a distal end and coupled to a proximal axial translation region at a proximal end. The proximal end may be configured to have multiple, in-line axial translation regions that each couple to a pull-wire or tendon so that axially moving the axial translation region relative to other regions of the device (e.g., pushing or pulling it longitudinally in the direction that the apparatus extends) may result in moving the pull-wire or tendon and bending the bendable distal region. The in-line axial translation regions may be connected to each other, e.g., elastically connected to each other via a spring or stretchable/compressible material.

In US 2014/0052109 it is described a steerable catheter probe having a body portion adapted for being connected to a proximal hub and a distal end portion connected to the body portion, wherein the catheter body defines a lumen; the distal end portion having a compressible segment and a non-compressible segment, the compressible segment having a longitudinal centerline; and a pull member attached to the distal end portion and adapted for applying a proximally directed force to the distal end portion whereby at least a portion of said compressible segment is compressed. Steering is provided by the positioning of the pull member and the design of the compressible segment: this comprises several compression coils, and at least one of its members of smaller diameter than the other larger and equal diameter coils. A pull wire is attached at a fixation point to one of the large diameter coils distal to the small diameter coil, and arranged within the lumen of large diameter coils but outside the lumen of any smaller diameter coils. Further examples of prior art steerable devices/guidewires are described for instance in U.S. Pat. No. 5,203,772, US 2014/0343538, US 2006/0241519, US 2001/0037084, US 2008/0027285 and US 2016/0206853.

All the above-cited prior art solutions present several shortcomings. For instance, many of the known steerable guidewires/catheters are bulky, with a complicated actuation system and resulting in complex manufacturing, which raises production costs and limit the miniaturization of the final product, that in some instances is critical for e.g. navigating in tortuous, tiny blood vessels as found in the brain vascular network. Additionally, many of the known devices can deflect in several directions, a feature that enhance even more the fabrication complexity and the operation of the device itself, mainly due to sophisticated actuation means. In this context, it should be mentioned that medical practitioners intended to use guidewires or catheters are generally quite reticent to drift from common clinical practice; the use of such complex tools would require a certain training for mastering their use, and this could hinder or at least limit the adoption of the technology. Furthermore, actuators are always located at the very proximal end of the guidewire; up to the inventors' knowledge, no existing systems provide a clutchable manipulator which can be fixed by the surgeon at any desired portion of a guidewire's body while still providing actuation means to the deflectable tip.

There is therefore still need for simple, not expensive and user-friendly steerable devices such as guidewires or catheters, which can be reliably miniaturized to commonly used dimension in IR while providing enhanced control for navigating in tortuous blood vessel network.

### SUMMARY OF INVENTION

Accordingly, the present inventors developed a device, system and methods for using thereof that address and overcome the drawbacks of the prior art solutions as summarized above.

In particular, the invention relates to a steerable device, embodied in some aspects of the invention as a guidewire for surgical procedures, and an associated system further comprising a handle with advanced features to allow a user to control the insertion and guiding of the device.

A first aim of the invention was to manufacture a steerable guidewire through a simple, reliable and cheap manufacturing process.

A second aim of the invention was to create a guidewire having steering properties which is easy to operate and manipulate so to access complex and tortuous vascular networks.

A further aim of the invention was to create a system comprising a steerable guidewire which can be used by an operator (such as a surgeon) by minimally drifting from the common clinical practice. All these aims have been accomplished by the present invention as defined hereinafter and in the appended claims.

A main consideration on which the invention is based is that devices for insertion into a subject's body do not need to bend or deflect in several axes, but on the contrary providing a single plane deflection would largely suffice in the context of interventional radiology, and could be actually preferred. As briefly summarized above, in clinical practice, a surgeon selects the size and the shape of guidewires depending on the needs and circumstances, and usually bends or pre-bends the distal, flexible end of the wire on one side in order to facilitate access thereof into tortuous paths. During this path finding procedure, performed with the help of imaging means, the user moves on the wire along a bodily cavity (such as a vessel) and twists it, often using a torquer device, according to the anatomy of said cavity in order to rotate the one-sided bent tip of the guidewire towards the selected vessel. Additionally, the imaging mean is adequately oriented perpendicular to the plane of the vessel intersection. As it can be understood, providing a single plane deflection guidewire, possibly actuated with user-friendly actuation means, would facilitate the directional positioning of the tip during operations while maintaining the common clinician's clinical practice, and avoiding the risks related to guidewire replacement or a tip re-shaping procedure.

It is therefore a first object of the present invention to provide for a steerable device having an elongated body defining a longitudinally-arranged lumen, said device comprising:
a) a proximal end portion adapted for handling by a user;
b) a distal end portion comprising a bendable portion and a tip; and
c) a pull wire connected to the distal end of the bendable portion and extending therefrom up to said proximal end along said lumen
characterized in that the bendable portion comprises a reinforcement structure positioned on one lateral side and a flexible, stress relief portion positioned on the opposed lateral side.

In one embodiment, the bendable portion is located at the very distal end of the device, and the pull wire is connected to the tip.

In one embodiment, the proximal end portion and/or the elongated body comprise an actuation region adapted to impart a force on the structure resulting in a distally-directed steering action on the bendable region. The actuation region is adapted to impart a tension force on the pull wire or an extension force on the elongated body resulting in a distally-directed force to the bendable region. In particular, the actuation region is adapted to impart a proximally-directed force on the pull wire or a distally-directed force to the bendable region.

In one embodiment, said actuation region comprises a coiled member.

In one embodiment, the reinforcement structure is integral part of the elongated body.

In one embodiment, the bendable portion is compressible.

In one embodiment, the bendable portion comprises a coiled member.

In one embodiment, the flexible, stress relief portion comprises a plurality of cut-outs located on one lateral side of the bendable portion.

In one embodiment, the device is configured as a guidewire for insertion into a subject's body.

Another aspect of the present invention relates to a system comprising the above-described device and an actuator adapted to impart force on the structure resulting in a distally-directed steering action on the bendable region, such as a tension force on the pull wire resulting in a distally-directed force to the bendable region. In particular, the actuator can impart a proximally-directed force on the pull wire or a distally-directed force on the bendable portion.

In one embodiment, the system is characterized in that the actuator is a torque device comprising:
a) a first handle comprising:
   an elongated body, a proximal end and a distal end, said handle defining a lumen along its entire length, and said distal end comprising means for gripping and ungripping the above-described steerable device;
b) a second handle comprising:
   an elongated body, a proximal end and a distal end, said handle defining a lumen along its entire length, and said distal end comprising means for gripping and ungripping the above-described steerable device;
wherein the first and second handles are arranged in a coaxial configuration so that they can perform a longitudinal relative displacement.

In one embodiment, said gripping and ungripping means comprise a spring collet clamp.

In one embodiment, said distal ends of said first and second handles each comprise a tapered tip adapted to engage said gripping and ungripping means and tighten them upon a longitudinal relative displacement of said first and second handles.

In one embodiment, the first and second handles are connected through an accordion-like, corrugated element.

In one embodiment, the first and second handles are connected through a spring clip.

In one embodiment, the first and second handles are arranged in a coaxial male-female configuration.

In one embodiment, each of said first and second handles comprises a plurality of corrugations on their inner lumen adapted to work as snaps.

In one embodiment, the system further comprises a first and second tapered fasteners adapted to releasably engage and tighten said gripping and ungripping means of said first and second handles, respectively.

In one embodiment, one of the first or second handle comprises a longitudinal slot along its body, and the other of the first or second handle comprises a protrusion adapted to fit and slide along said longitudinal slot.

In one embodiment, the system further comprises a gear rotational actuator adapted to engage said protrusion and operate upon rotation a longitudinal relative displacement of said first and second handles.

### BRIEF DESCRIPTION OF DRAWINGS

In the Figures:

FIGS. 1A-1D depict one embodiment of the device of the invention: 1A) overview of the device; 1B) lateral view; 1C) bottom view; 1D) longitudinal cross-section;

FIGS. 2A-2D depict two alternative embodiment of the device of the invention: 2A) and 2B) a lateral and longitudinal cross-section of the device having the inner pull wire connected to the distal end of the bendable portion; 2C) and 2D) a lateral and longitudinal cross-section of the device having the inner pull wire connected to the tip, wherein the bendable portion is located proximally compared to said tip;

FIGS. 3A and 3B depict two lateral views of the device in a rest and actuated positions; notice the compression of the stress relief elements (coils) on the inner side of the bend, and the constriction of the coils on the outer side of the bend;

FIGS. 4A-4D show four alternative embodiments of the reinforcement structure: 4A) rectangular; 4B) triangular; 4C) rhomboidal; 4D) “butterfly” shape;

FIGS. 5A-5C show an alternative embodiment of the device of the invention comprising a segmented reinforcement structure: 5A) lateral view and 5B) bottom view of the device in a rest position; 5C) lateral view of the device in an actuated position, showing elbow bends;

FIG. 6 shows a longitudinal cross section of an alternative embodiment of the device of the invention, characterized in that at least the bendable portion is composed of a solid body made for instance of a metal or a soft, polymeric material, said body having a plurality of cutouts defining the stress relief portion;

FIGS. 7A-7F show alternative embodiments of the device of FIG. 6, in which the cutouts have a triangular (7A) and (7D), rounded (7B) and (7E) or rectangular (7C) and (7F) appearance;

FIGS. 8A and 8B show two longitudinal cross sectional views of the actuation portion in a rest and actuated positions;

FIG. 9 depicts an overview of one embodiment of a system according to the invention, comprising the steerable device and an actuator;

FIGS. 10A and 10B show one embodiment of the actuator, having two handles connected via an accordion-like element;

FIGS. 11A-11D show two embodiments of the actuator, having two handles connected via 11A) and 11C) a spring and a displacement element or 11B) and 11D) a spring tab or clip;

FIGS. 12A and 12B show one embodiment of the actuator: 12A) exploded view and 12B) isometric view;

FIGS. 13A-13C show a longitudinal cross sectional view of the actuator of FIGS. 12A and 12B, as well as of the fasteners;

FIGS. 14A and 14B show a partial longitudinal cross sectional view of one embodiment of the actuator comprising two handles interacting by means of snaps;

FIGS. 15A and 15B show one embodiment of the actuator comprising a gear rotational element: 15A) exploded view and 15B) isometric view;

FIGS. 16A and 16B show a longitudinal cross sectional view of the actuator of FIGS. 15A and 15B both in a rest position (16A) or in operation (16B);

FIGS. 17A and 17B show a comparison between state-of-the-art, conventional pre-bent guidewires known by the prior art (17A) and the device of the invention (17B); notice the ability of the steerable guidewire to reach sub-arteries not reachable with classic guidewires;

FIGS. 18A and 18B depict the actuator-driven actuation of the device of the invention, in which a distally-directed force imparted on a coiled actuation region of the device allows deflection thereof.

### DESCRIPTION OF EMBODIMENTS

The present disclosure may be more readily understood by reference to the following detailed description presented in connection with the accompanying figures, which form a part of this disclosure. It is to be understood that this disclosure is not limited to the specific conditions or parameters described and/or shown herein, and that the terminology used herein is for the purpose of describing particular embodiments by way of example only and is not intended to be limiting of the claimed disclosure.

As used herein and in the appended claims, the singular forms “a”, “an” and “the” include plural referents unless the context clearly dictates otherwise. Also, the use of “or” means “and/or” unless stated otherwise. Similarly, “comprise”, “comprises”, “comprising”, “include”, “includes” and “including” are interchangeable and not intended to be limiting. It is to be further understood that where descriptions of various embodiments use the term “comprising”, those skilled in the art would understand that in some specific instances, an embodiment can be alternatively described using language “consisting essentially of” or “consisting of.”

The present invention is directed to a steerable device capable of bending, deflecting, or otherwise being controlled off-axis, which is some instances is embodied as a guidewire adapted to move within a certain target location such as a vessel, body tissue or otherwise hollow organ.

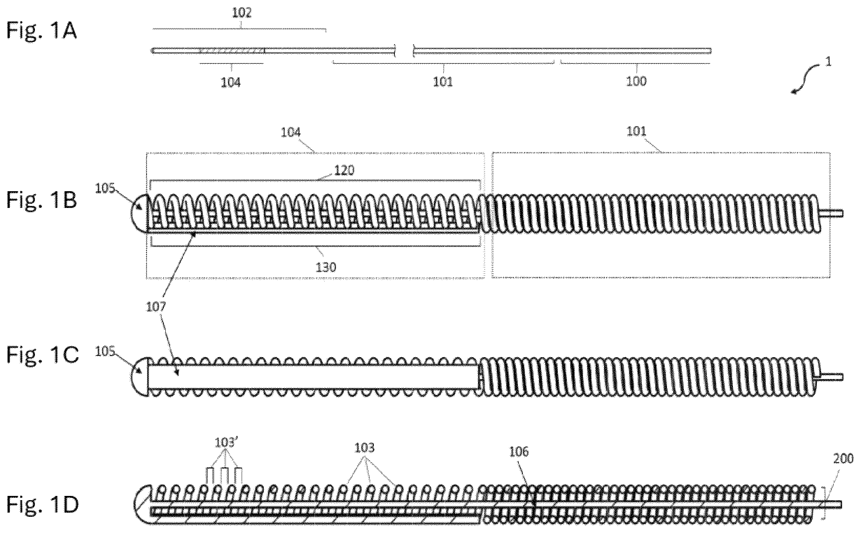

With reference to FIGS. 1A-1D, one embodiment of the device of the invention is shown. Device 1 comprises a proximal end 100, an elongated body 101 and a distal end portion 102. A lumen 200 spans from the distal end 102 up to the proximal end 100 and along the entire body 101, said lumen being coaxially arranged compared to the longitudinal axis of the device. The distal end portion 102 comprises a bendable portion 104 and a tip 105 at its very end. Tip 105 is generally an atraumatic and non-sharp tip, with a rounded, oval, or similar appearance. Materials opaque to X-rays, such as platinum, gold, tungsten, tantalum or the like, may be advantageously incorporated therein, to act as a fluoroscopic marker to aid in visualization. The bendable portion 104 is a region at the distal end 102 of the device that is significantly more flexible and susceptible to deflection than the remaining body 101 and/or proximal region 100.

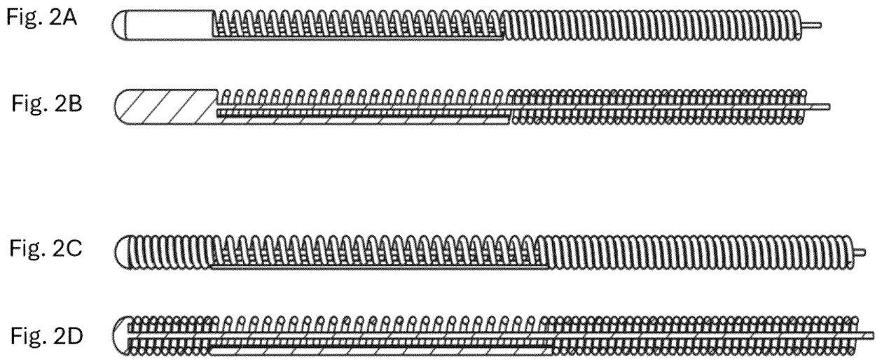

A pull wire 106 is affixed to the distal end of the bendable portion 104 and extends therefrom up to said proximal end 100 along said lumen 200. In the depicted embodiment, the bendable portion 104 is located at the very distal end of the device, and the pull wire 106 is connected to the tip 105; alternative embodiments are shown on FIGS. 2A-2D. The device is characterized by the fact of comprising, in the bendable portion 104, a reinforcement structure 107 advantageously located on one lateral side and a flexible, stress relief portion 120, located on the opposed lateral side.

The steerability, deflection or bending of a distal region 102 of the guidewire device, in particular of the bendable portion 104 thereof, is obtained by imparting a longitudinal tension force on the pull wire 106. For instance, this can be accomplished by using the inner pull wire 106, longitudinally arranged with respect to the device's body inside the lumen 200 thereof, and free to translate along the device longitudinal axis. Said pull wire 106 is axially rigid while being flexible, and a translational motion of said wire 106 relatively to the lumen 200 along the longitudinal axis, through application of a tensile strength in a proximal direction, is used to generate the deflection. In another scenario, however, the deflection of the bendable portion 104 can be obtained by applying a distally-directed force on it when the pull wire 106 is further affixed at the proximal end 100, as it will be detailed later on.

The steerable device is fabricated so that it is substantially straight from its proximal end 100 to its distal end 102. As will be explained later on in more details, manipulation of a control mechanism at the proximal end 100 and/or body 101 causes the distal, bendable region 104 of the steerable device to bend or curve away from its longitudinal axis.

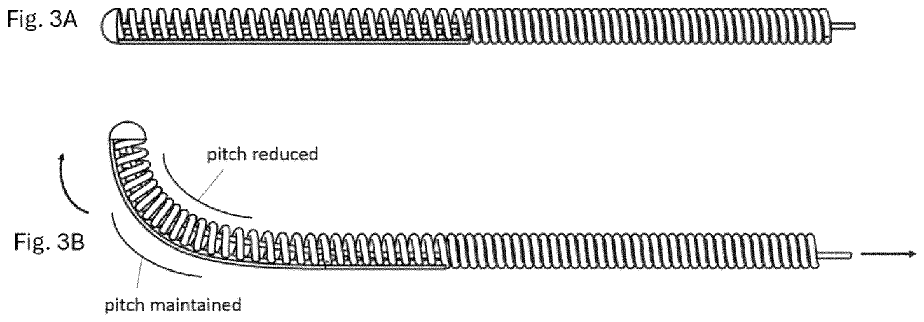

The bendable region 104 is characterized by the presence of two distinct portions, each positioned on one side of said region 104, and one opposed to the other, that are named herein for the sake of clarity and simplicity stress relief portion 120 and constrained portion 130. Those two portions are designed to induce an asymmetric compression in the bendable region 104 that guides the direction of bending when an axial force (stress) is applied thereon by the pull wire 106. To do so, the stress relief portion 120 comprises a series of stress relief elements 103 separated by hollow spaces 103′ that allows for the compressibility of the bendable region 104; on the contrary, the constrained portion 130 is characterized by the presence of a so-called reinforcement structure 107 that limits the compressibility of one side of said bendable region 104 so that the generation of a compression stress will induce a bending which is opposed to the side of the reinforcement structure 107, as schematically depicted in FIGS. 3A and 3B. As it will be therefore apparent, the device of the invention is characterized by the fact of being bendable or otherwise deflectable on a single side only.

In one embodiment, the entire device or portions thereof can comprise or consist of a coiled member. In particular, the distal end portion 102, particularly the bendable portion 104 of this latter, and the proximal end 100 and/or the device body 101 can comprise or be formed as a helix or a coil. In preferred embodiments, the bendable portion 104 can comprise or be formed as a helix or a coil having a finite spacing (103′) between the coils (103) or windings. As a result of this configuration, the bendable portion 104 mechanically results as a loosely wound spring coil, so that adjacent windings or coils 103 are not in contact with one another in the absence of any tensile stress. As is typically the case for guidewires designed to navigate vessels of humans and animals, and used as a guide for e.g. placement of a catheter, the proximal end 100 and the body 101 are formed by a tightly wound coil of a high tensile strength wire of a resilient, noncorrosive metal such as stainless steel, nitinol, platinum or other biocompatible materials, as well as any combination of the foregoing. This arrangement stiffens the device's shaft except for the distal end, providing sufficient column strength so that it can advantageously be pushed, from the proximal end 100, to force the distal portion 102 through the vasculature of a patient, while said portion 102 is flexible enough to be deflected by blood vessel wall so that it may impinge on the blood vessel walls during passage without deforming, puncturing or injuring the blood vessel. Thus, referring to FIGS. 3A and 3B, it can be seen that a lateral deflection of steering region (i.e., the bendable portion 104) to one side may involve an axial compression of adjacent wire loops (pitch) on the inside curve (stress relief portion 120) of the bend, while constraining or otherwise limiting the pitch on the outside curve (constrained portion 130) of the bend.

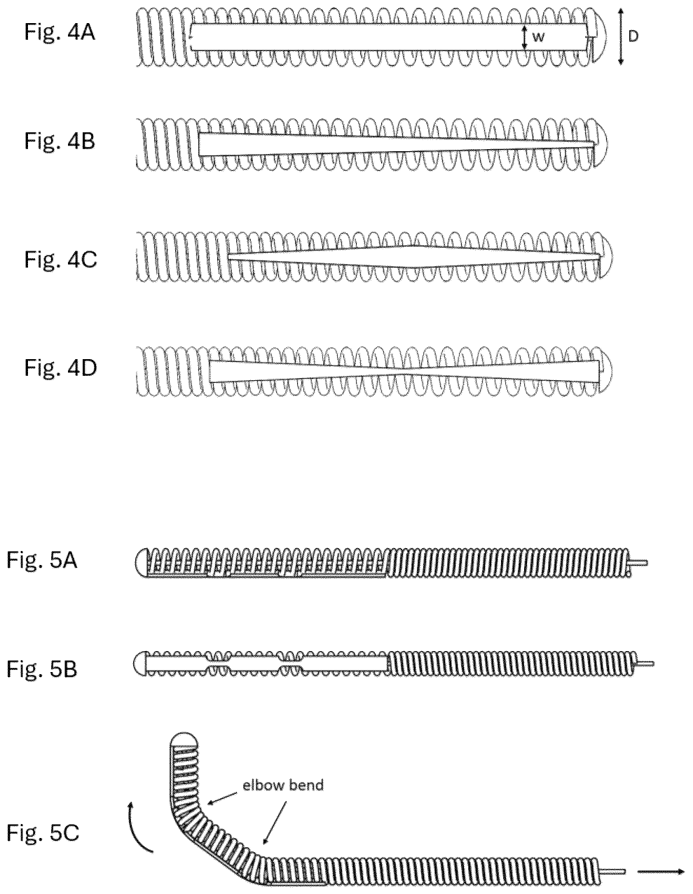

In some embodiments, the reinforcement structure 107 can have various different shapes, and even more than one reinforcement structures 107 can be present along the bendable portion 104. Moreover, in some embodiments the reinforcement structure 107 can span from the distal tip 105 all along the bendable portion 104, so that a constrained portion 130 would cover one entire side thereof, or the reinforcement structure 107 can define a more limited portion of bendable portion 104 as a constrained portion 130. As depicted in the various embodiments in FIGS. 4A-4D, the reinforcement structure 107 may have e.g. a rectangular, triangular, rhomboidal or any other suitable contour appearance depending on the needs and circumstances; the contour shape would define the resistance to the bending of the constrained portion 130 upon application of a tensile stress, which will be higher in those portions of the constrained region 130 having larger width of the reinforcement structure 107 and vice versa, assuming an homogeneous thickness of the reinforcement structure. Moreover, as depicted e.g. in FIGS. 5A-5C, more than one reinforcement structure 107, or a sectioned one, can be implemented on the bendable portion 104 in order to set a plurality of constrained portions 130. This embodiment is particularly useful for obtaining “elbow” deflections of the bendable portion 104, wherein by “elbow” deflection it is meant that the bend in the bendable portion 104 does not occur in a uniform way along the bendable portion 104 but at a relatively discrete position(s) displaced proximally from the tip 105 of the guidewire. This arrangement could enable or facilitate negotiation of the artery with e.g. minimal trauma to the vascular intima.

Typically, the bendable portion 104 is smaller or equal in length than the reinforcement structure 107 length, such as about from 5% to 105% of reinforcement structure 107 length. A typical range for the width of reinforcement structure w is 5% to 95% of the bendable portion 104 diameter, D. The reinforcement structure 107 profile more preferably follows the curvature of the coiled member structure when observed through an axial view facing a cross section. Moreover, a typical range for coil spacing in the bendable portion 104 is +5% to 1000% of the coil spacing in a coiled body region 101.

In some embodiments, the reinforcement structure 107 is substantially made of an elastic and/or stretchable material such as for instance various elastomers, silicon polymeric materials like Polydimethylsiloxane (PDMS), silicon adhesives, silicone rubbers, natural rubbers, stretchable fabric, thermoplastic elastomers, hydrogels, polyamide, polyimide. In some additional or alternative embodiments, the reinforcement structure 107 is substantially made of non-elastic, stiff materials such as for instance poly ethylene (PE), poly propylene (PP), polyether etherketone (PEEK), Acrylonitrile butadiene styrene (ABS), epoxys, polytetrafluoroethylene (PTFE), polyurethane, thermoplastic polyurethanes (TPU), Nylon, Polyether block amide (PeBax), kevlar and liquid-crystal polymers or metals such as for instance stainless titanium, steel, nickel titanium alloy (Nitinol), tungsten, cobalt, chrome, nickel, aluminium, copper, molybdenum or any combination thereof. This embodiment is particularly suitable and advantageous in situations when an elbow deflection is sought, as shown in FIGS. 5A-5C.

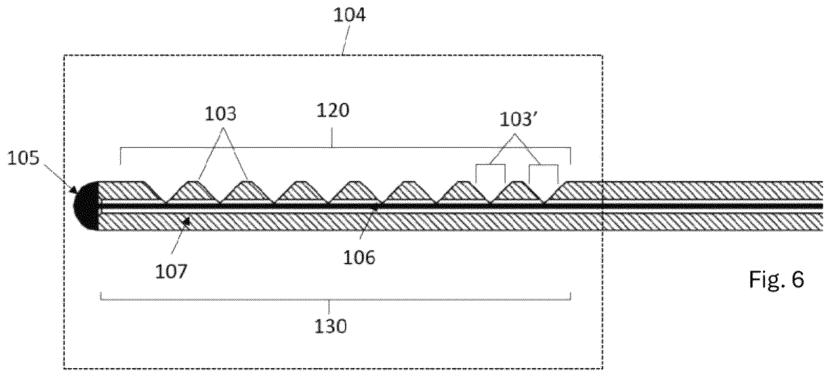

In another embodiment according to the invention depicted in FIG. 6, the device is composed, at least at its distal end 102 and particularly at the bendable region 104, of a solid body, such as a soft, polymeric body, containing a thru hole defining the lumen 200 in which a pull wire 106 is inserted. The pull wire 106 is attached by any suitable means such as for instance gluing on the distal end tip 105 of the solid body. The bendable region 104 of the solid body, which can have any lateral cross-section (perpendicular to the longitudinal axis) such as round, elliptic, square, rectangular and the like, comprises a plurality of stress relief elements 103 in its stress relief portion 120 embodied as cutouts. In embodiments where the elongated body 101 of the device is also made of the same material of said solid body, such as a polymeric material or a metal, the reinforcement structure 107 can constitute an integral part of said body 101. Briefly, a tubular polymeric element can be shaped to include a plurality of cutouts on one side of one of its extremities while leaving the other side unaltered, so to define a built-in blocked lateral side 130 and a lateral bendable side 120.

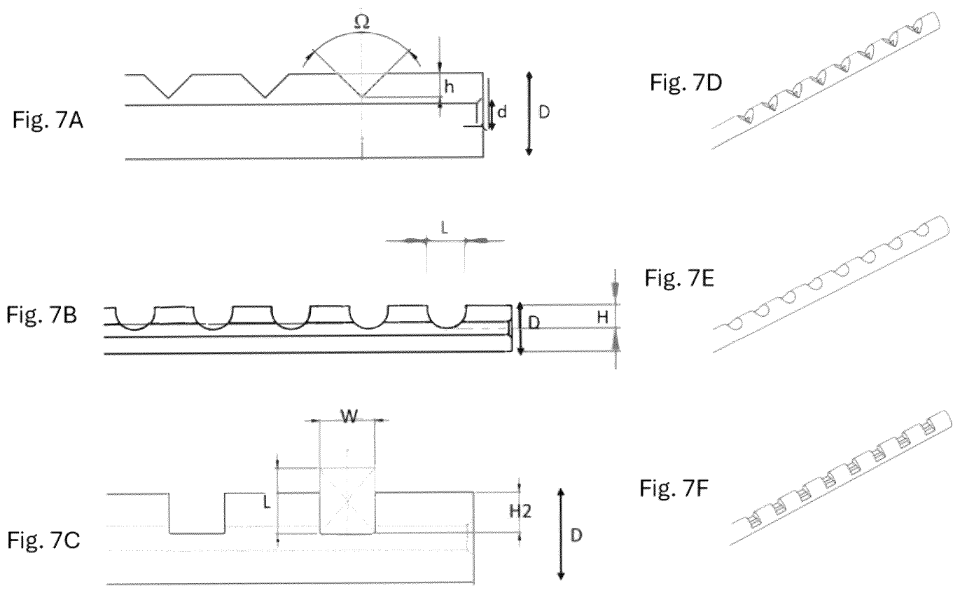

In the embodiment represented in FIG. 6, the distal end 102 of a soft polymeric element is rendered single-sided deflectable by cutting slots or gaps 103′ generally having hemispherical, rounded, trapezoidal, square or triangular profile, those gaps 103′ not passing completely through the entire thickness of the polymeric element so that a sort of spine with ribs is formed. As better shown in FIGS. 7A and 7D depicting a triangular cutouts embodiment, the ratio between the guidewire inner diameter d and the guidewire outer diameter D is typically comprised between 1:10 and 1:1.1, the cutout angle (2 ranges from about 5° to 165°, typically from 30° to 120°, and the cutout height h ranges from about 5% to 95% of D, typically from 10% to 50% of D. In a rounded cutouts embodiment (FIGS. 7B and 7E), a typical cutout height H ranges from about 5% to 95% of D (starting from central axis), typically from 50% to 75% of D, with an L to H ratio comprised between 0.01 and 10. In a rectangular cutouts embodiment (FIGS. 7C and 7F), a typical ratio between the height of rectangular cutout L and the width of rectangular cutout W ranges from 1:1 to 1:8, and a typical cutout height H2 ranges from about 5% to 95%, typically from 10 to 50% of D, with L≥H2. The number of cutouts typically ranges from 1 to 300, and the bendable region 104 can span e.g. from 1 mm up to 10 cm.

A steerable guidewire according to the invention can be fabricated to have diameters ranging from about 0.007 inches to about 0.038 inches. The external diameter of wire wound guidewires will of course be a function of the intended application. For instance, a steerable guidewire according to the invention intended for coronary angioplasty is preferably wound to have an external diameter in the range of from about 0.014″ to about 0.018″.

The device of the invention embodied as a guidewire can come in various stiffnesses, and its length can range from about 50-cm to about 350-cm or longer. Typical guidewire diameters for cardiovascular use have diameters in the range of 0.007″ to 0.018″ to 0.025″, a length in the range of 100 to 300 cm, and a tip stiffness of 1 to 10 grams for access to unoccluded vessels, and 10 to 30 grams for crossing occluded or stenosed vessels. Tip stiffness is measured by the amount of force/weight needed to deflect the tip by 45%. Peripheral guidewires are larger in diameter (in the range of about 0.032″, 0.038″, or greater) and may have a higher tip stiffness. A key advantage of the inventive concept behind the invention relies on the positioning of a reinforcement structure 107 that minimally alter the entire structure of a guidewire, rendering even very thin wires adapted to deflect without the need of bulky apparatuses or actuation mechanisms.

For some applications, such as coronary access, the distal end 102 will be floppier (have a lower stiffness) than the body 101 of the guidewire, while in other applications, such as aortic access, the distal end 102 elongated body 101 may have substantially similar stiffness. The distal end 102 may extend 0.5 to 10 cm, such as 1 to 5 centimeters.

In some embodiments, the exterior surface of the device is entirely or partially equipped with an elastic, biocompatible coating or sheath to provide a smooth outer surface. Suitable coatings can be formed by dipping, spraying or wrapping and heat curing operations as are known in the art. A coating material is selected to minimize sliding friction of the device during insertion and removal into a subject's body, and is substantially chemically inert in the in vivo vascular environment. A variety of suitable materials are known, including, for example, polytetrafluoroethylene (PTFE), tetrafluoroethylene (TFE), urethane, polyurethane, thermoplastic polyurethanes (TPU), silicone Polyether block amide (PeBax), Nylon or polyethylene.

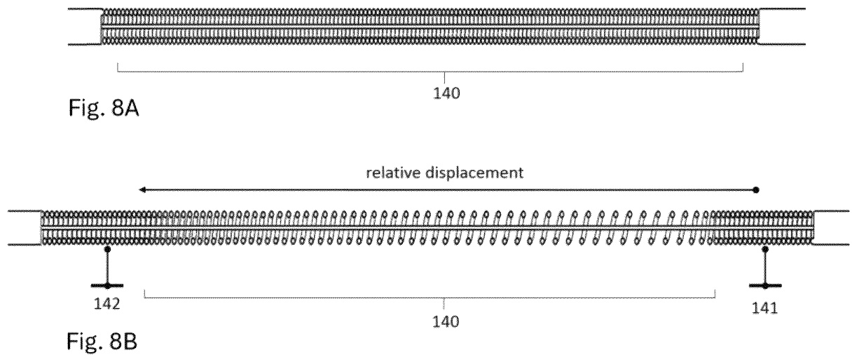

The steerable device or guidewire of the invention further comprises an actuation region 140 at its proximal end 100 and/or anywhere else along the body 101 of the device which is adapted to impart a tension force on the pull wire 106 resulting in a bending of the region 104, such as a proximally-directed force on the pull wire 106 or a distally-directed force on the bendable region 104, in order to control the articulation at the distal end 102, particularly at the bendable region 104. The actuation region 140 is designed to permit physical access to the pull wire 106 and manipulation thereof; for instance, the actuation region 140 can be placed at the very proximal end extremity of the device where the proximal end of the pull wire 106 can be directly clamped, grasped or otherwise manipulated to impart longitudinal forces thereon. Direct access to the pull wire 106 can however be attained anywhere along the proximal region 100 and/or the elongated body 101. In preferred embodiments, the actuation region 140 is elastic in nature, and can comprise or consist of a coiled member, as depicted in FIGS. 8A and 8B. In the shown scenario, the device is embodied as a guidewire having its body 101 formed by a tightly wound coil of a high tensile strength metal wire, a pull wire 106 affixed distally on the tip 105 and proximally to the proximal end 100, and the steering mechanism of action consists of a collapse of the bendable region 104 by means of longitudinal stress forces applied on the actuation region 140. In particular, the coiled portion of the actuation region 140 gets blocked, e.g. by clamping, on two opposed, longitudinally-placed anchor points 141 and 142. The relative displacement of the blocking point 141 and 142 imparts an axially, longitudinally directed force on the entire body 101 up to the distal end 102, which tends to collapse the hollow spaces 103′ in the stress relief portion 120 of the bendable region 104, finally bringing the stress relief elements 103 closer together.

Especially in embodiments of the invented device where a guidewire is envisaged, the actuation mechanism can be favoured by the use of an actuator adapted to impart a proximally-directed force on the pull wire 106 or a distally-directed force on the bendable portion 104.

This is mainly due to the physical and mechanical nature (e.g. a smooth surface), as well as the size (e.g. small diameter), of guidewires conventionally used in clinical practice, which renders their manipulation hard to be performed by hands. In this contexts, medical practitioners are used to operating guidewires with the help of small devices adapted to favour the grip and the torque of said wires, thus facilitating manoeuvrability thereof. A so-called torque device is used to provide a “handle” whereby the surgeon can have maximum control over the positioning and orientation of the guidewire.

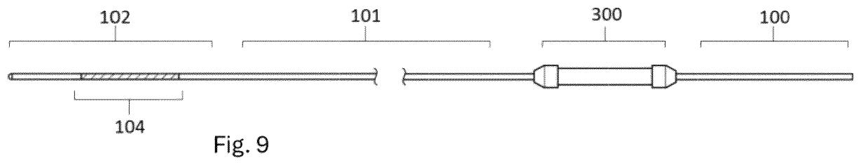

Accordingly, a further object of the present invention relates to a system comprising the device 1 of the invention and an actuator adapted to impart a proximally-directed force on the pull wire 106, or a distally-directed force on the bendable portion 104. The actuator, also referred to herein as “torque device”, “torque actuator” or “torquer”, features a controlling mechanism for permitting the articulation at the distal end 102 of the device of the invention, and can advantageously be releasably affixed to the proximal end 100 and/or body 101 of this latter to follow the advancement of the device along a path, such as the endoluminal space of a blood vessel, and allowing negotiation, pushing/pulling and steering operations (FIG. 9).

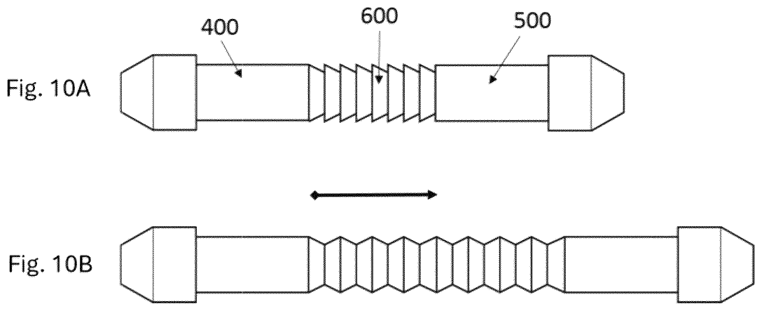

In the implementation of the invention, a plurality of non-limiting embodiments of said actuator 300 have been developed, depicted mainly in FIGS. 10A to 16B. Generally speaking, the developed torque actuator 300 comprises:
a) a first handle 400 comprising:
   an elongated body 401, a proximal end 402 and a distal end 403, said handle 400 defining a lumen 404 along its entire length, and said distal end 403 comprising means 410 for gripping and ungripping the above-described steerable device 1;
b) a second handle 500 comprising:
   an elongated body 501, a proximal end 503 and a distal end 502, said handle defining a lumen 504 along its entire length, and said distal end 502 comprising means 510 for gripping and ungripping the above-described steerable device 1;
wherein the first and second handles 400 and 500 are arranged in a coaxial configuration so that they can perform a longitudinal relative displacement.

A torquer 300 according to the invention securely holds and controls a guidewire with one hand and with smooth operation. The actuator is simple in its construction, fast to load and adjust, easily gripped and ungripped and free of jerking wire movements during operating procedures.

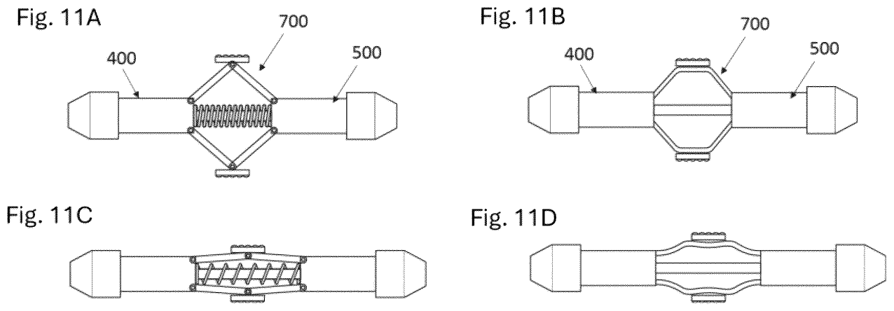

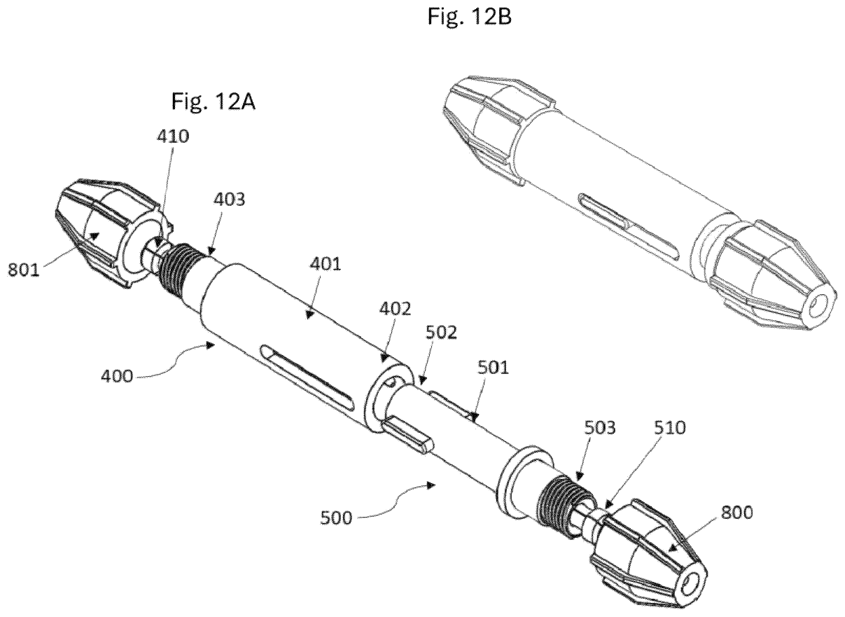

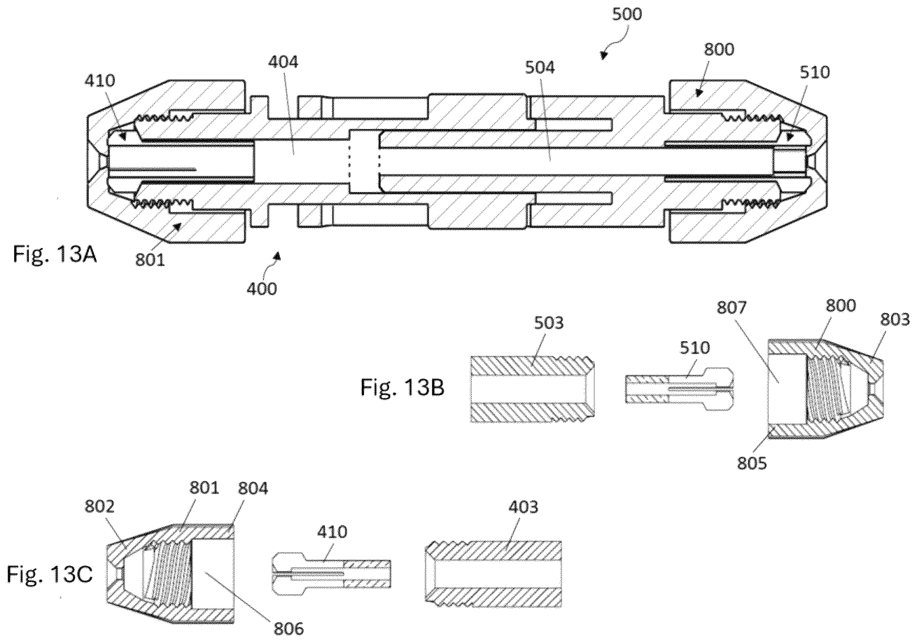

The torquer 300 is composed of two handles 400 and 500 connected or connectable through a variety of solutions; as a way of example, said handles 400 and 500 can be coupled and/or fixed one with respect to the other by means of an accordion-like, corrugated element 600 (FIGS. 10A and 10B), via a spring clip 700 (FIGS. 11A to 11D), or by a male-female arrangement, permitting an insertion of e.g. handle 400 into handle 500 (FIGS. 12A and B; FIGS. 13A to 13C). Notwithstanding the connection setup, handles 400 and 500 are substantially coaxially arranged one with respect to the other along their longitudinal axes.

As exemplary depicted in FIGS. 12A and B and FIGS. 13A to 13C, an interior long axial channel or lumen (404, 504) extends between the proximal end (402, 503) and the distal end (403, 502) through the elongated body (401, 501) of each of the first and second handles 400 and 500. An aperture formed at the proximal end (402, 503) and an aperture formed in the distal end (403, 502) provide access to the passage, and facilitate advancement, of a guidewire 1 through body. Channels or lumens 404 and 504 are substantially coaxially arranged one with respect to the other along their longitudinal axes so to create, upon connection of handles 400 and 500, a unitary passage or channel.

In preferred embodiments, gripping and ungripping means 410 and/or 510 comprise a spring collet clamp. A collet locking system comprises a chuck that forms a collar around an object to be held (in the present case, guidewire device 1) and exerts a strong clamping force on said object when it is tightened. The collet is a sleeve with an inner surface normally matching that of a guidewire and a conical outer surface. The collet can be squeezed against a matching taper such that its inner surface contracts to a slightly smaller diameter, squeezing the guidewire to hold it securely. This is achieved with a spring collet, made of e.g. spring steel, with one or more kerf cuts along its length defining at least two flanges that allow the spring collet to expand and contract. With enough compression, the collet locking mechanisms 410 and 510 exert sufficient frictional force upon the guidewire 1 such that this is “locked” relative to torque device 300, preventing the guidewire from sliding freely within the hollow inner bores 404 and 504 of torque device 300.

To perform said compression, in one embodiment the system further comprises a first and second tapered fasteners 800 and 801 adapted to releasably engage and tighten said gripping and ungripping means 410 and/or 510, such as a spring collect clamp, of said first and second handles 400 and 500, respectively. As shown for instance in FIGS. 12A-12B and 13A-13C, fasteners 800 and 801 are caps including a first end (802, 803) and a second end (804, 805), with a hollow passage extending therebetween (806, 807). An aperture formed in the first end (802, 803) and an aperture formed in the second end (804, 805) provide access to the passage and facilitate advancement of a guidewire 1 through the cap (800, 801). Hollow passages 806 and 807 are substantially coaxially arranged with respect to channels or lumens 404 and 504 along their longitudinal axes so to create, upon connection with handles 400 and 500, a unitary passage or channel. The first end (802, 803) is tapered so that, upon connection with distal end (403, 502) of a handle (400, 500), the spring collect clamp (410, 510) is locked against a wire inserted therein. Threads formed adjacent the second end (804, 805) on an interior of the fastener (800, 801) can cooperate with threads at the distal end (403, 503) of a handle (400, 500).

In one additional or alternative embodiment, said distal ends 403 and 503 of said first and second handles 400 and 500 each comprise a tapered tip adapted to engage said gripping and ungripping means 410 and/or 510 such as a spring collect clamp and tighten them upon a longitudinal relative displacement of said first and second handles 400 and 500. In this embodiment, a collet clamp 410 or 510 is designed as a tubular element coaxially arranged along its longitudinal axis within the hollow elongated body of the handle (401 or 501). The diameter of the collet (410, 510) matches the bore (401, 501) of the handle (400, 500), having the larger, distal tapered end slightly greater than the bore while the smaller, proximal diameter is slightly less than the bore. A longitudinal relative displacement of the first and second handles 400 and 500 pushes the distal ends 403 and 503 thereof against the distal, larger end of the collet 410 and 510, forcing the tapered distal end of the collet clamp (410, 510) to slide within the hollow elongated body of the handle (401 or 501), thus locking the two elements together. Upon release of the force necessary to perform the relative displacement of handles 400 and 500, the elastic spring nature of the collet (410, 510) allows this latter to be pushed outside the body (401,501), thus consequently releasing the clamping force. As it will be evident to a person skilled in the art, combinations of the above-described embodiments are also envisageable; for instance, in operation, the first handle 400 is proximally arranged compared to the operator, while the second handle 500 is distally located. The operator can fix a proximally-located anchorage point 141 by screwing the fastener 800 about the threaded distal end 403 of handle 400, to tighten the collet 410 and apply a clamping force on the device 1. In this way, the actuator 300 can be operated as a torquer conventionally used in clinical practice; however, the distal end 503 of distally-located handle 500 can be tapered as to engage the collet 510 and automatically clamp and tighten it on an anchorage point 142 upon a longitudinal relative displacement of said first and second handles 400 and 500.

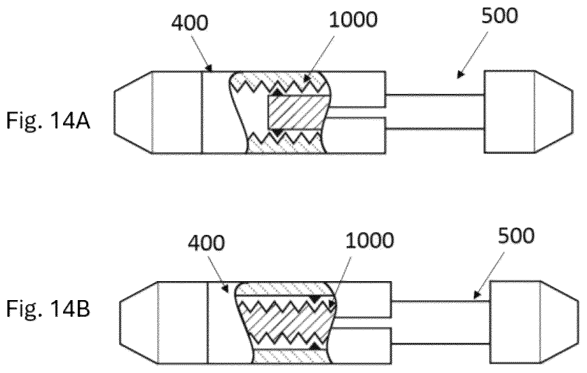

Moreover, in a further embodiment according to the invention, each of said first and second handles 400 and 500 can comprise a plurality of corrugations 1000 on their inner lumen adapted to work as snaps. In this way, handles 400 and 500 can be fixed one relative to the other in defined positions depending on the needs (FIGS. 14A and 14B). This feature is particularly advantageous for providing a tactile feedback to the operator, and to facilitate the incremental actuation of the handles' displacement-acting in turn on the incremental deflection of the device's distal end-especially with actuators as those depicted in FIGS. 10A to 16B.

Preferably, handles 400 and 500 are designed for impeding or at least limiting their relative rotation with respect to each other as much as possible. Advantageously, handles 400 and 500 are longitudinally constrained by e.g. a guiding mechanism having grooves and guided pins, or via matching slots designed on the inner surface of their lumens.

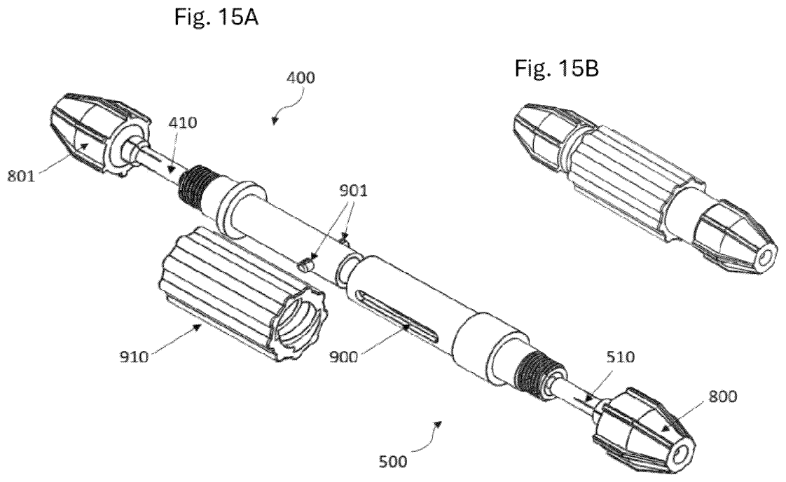

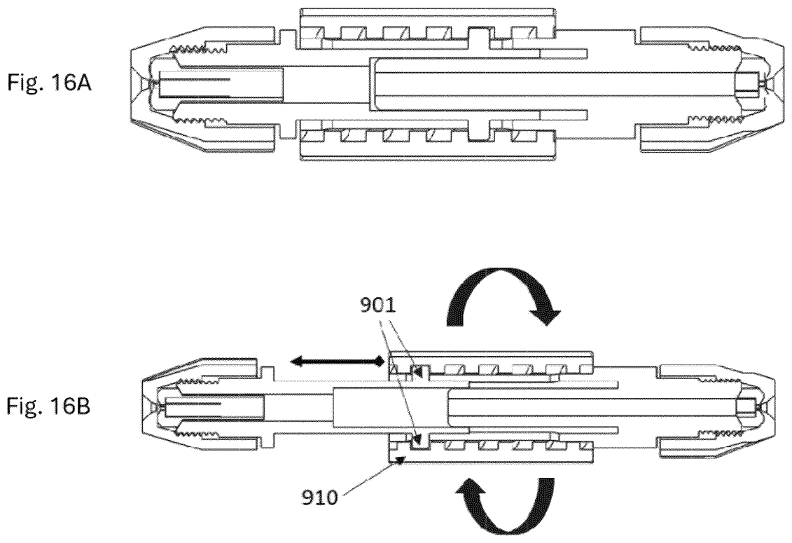

In still another embodiment, one of the first or second handle 400 or 500 comprises a longitudinal slot 900 along its body, and the other of the first or second handle 400 or 500 comprises a protrusion 901 adapted to fit and slide along said longitudinal slot 900. In this context, the system further comprises a gear rotational actuator 910 adapted to engage said protrusion 901 and operate, upon rotation, a longitudinal relative displacement of said first and second handles 400 and 500 (FIGS. 15A-15B and 16A-16B).

As a way of example, one method of use involves performing a percutaneous or cutdown procedure to gain access to structures such as, but not limited to, the vasculature, either a vein, an artery, a body lumen or duct, a hollow organ, musculature, fascia, cutaneous tissue, the abdominal cavity, the thoracic cavity, and the like. An introducer, which is usually a hollow, large diameter, hypodermic needle, and the steerable guidewire are placed within the vasculature and the steerable guidewire is advanced through the central lumen of said introducer to be routed proximate to the target treatment site. The introducer can be removed at this time or substantially at the time the guidewire is introduced into the body lumen.

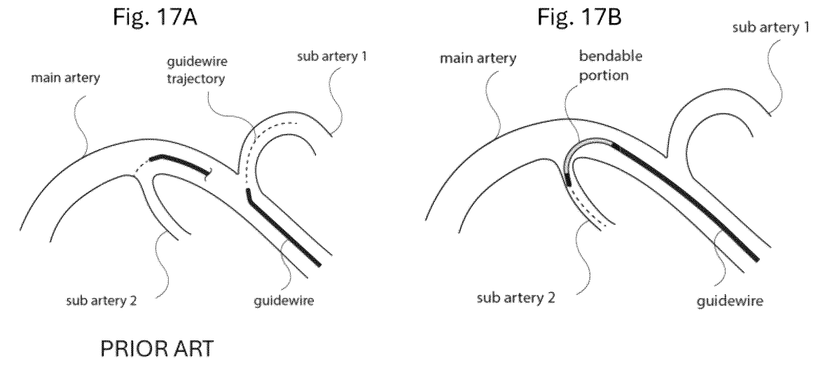

The torque device 300 is assembled or otherwise arranged so that the steerable guidewire 1 is fed within the unitary channel formed by channels 404 and 504, collet clamps' bores 410 and 510 and, whenever present, hollow passages 806 and 807 of the fasteners 800 and 801, as well as through the apertures of the foregoing. Deflection of the distal end 102 to varying degrees of curvature, under control from the proximal end 100 or body 101 of the guidewire can be performed. The curve can be oriented along the direction of a branching vessel or vessel curve so that the steerable guidewire can then be advanced into the vessel by way of its high column strength and torqueability (FIGS. 17A and 17B).

For instance, when a branch or bend in the body vessel is reached, the first and second handles 400 and 500 are rotated and threaded into the second ends 804 and 805 on an interior of the fasteners 800 and 801 (or vice versa), so that the handles' distal ends 403 and 503 are advanced into the narrowing passage of the fasteners 800 and 801 towards the tapered portions thereof 802, 803. As distal ends 403, 503 and first ends 802, 803, respectively, converge, the flanges of spring collet clamps 410 and 510 are compressed around the guidewire to produce sufficient frictional force to grasp the guidewire, thereby locking it relative to the torque device 300. In this configuration, the torque device 300 is therefore in a closed position and advancement of the guidewire through it is impeded. The user can exploit the torque device 300 as a handle to easily manipulate the guidewire (e.g. twisting and/or laterally move it).

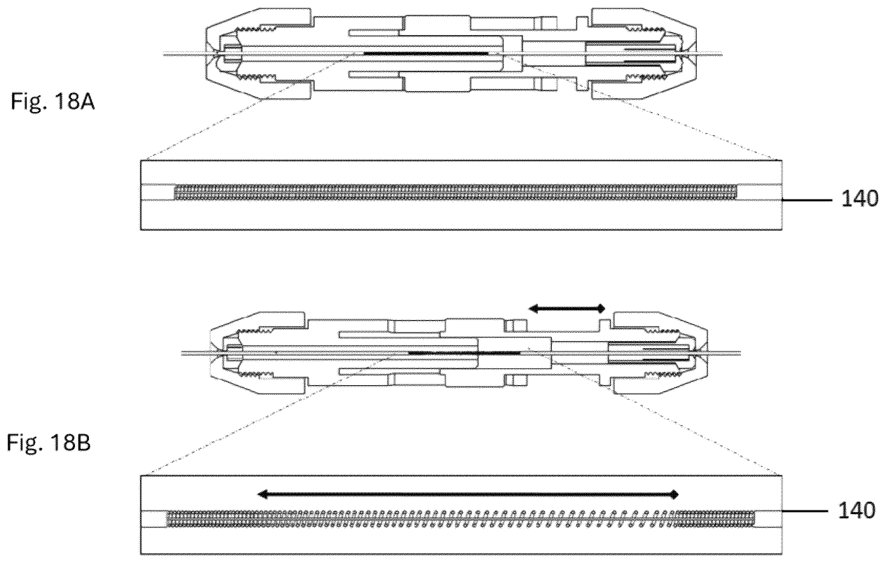

At this point, a relative displacement between first and second handles 400 and 500 can be performed by the user (FIGS. 18A and 18B). A connection element between said first and second handles 400 and 500, such as an accordion-like, corrugated element 600 (FIGS. 10A and 10B), a spring clip 700 (FIGS. 11A to 11D) or longitudinal slot 900/protrusion 901 coupled by a gear rotational actuator 910 (FIGS. 15A-15B and 16A-16B), is operated by the user to move away first and second handles 400 and 500 along their common longitudinal axis. The torque device 300 is anchored on the proximal end 100 or the body 101 of the guidewire on two anchor points 141 and 142 via its first ends 802 and 803, the displacement of the handles on the designated actuation region 140 imparts a distally-directed force on the bendable portion 104, that in turn collapses the hollow spaces 103′ in the stress relief portion 120 of the guidewire's bendable region 104, bringing the stress relief elements 103 closer together and permitting the device's bending towards the target vessel or capillary. The surgeon can also release the stress tension when convenient. To do so, the first and second handles 400 and 500 are moved back in the initial position by acting on the connection element (e.g., spring clip 700 or gear rotational actuator 910). The fasteners 800 and 801 can be, at this point, unscrewed from distal ends 403 and 503 to allow the guidewire to be fed through the torquer 300. Once the guidewire is in the desired location and advancement through the body vessel is complete, the torque device 300 may be opened and slidably removed from the guidewire by its proximal end. Once the guidewire is in place, the surgeon can further proceed with the endovascular procedure.

1. A steerable guidewire having an elongated body defining a longitudinally-arranged lumen, said steerable guidewire comprising:
a) a proximal end portion;
b) a distal end portion comprising a bendable portion and a tip, said bendable portion comprising a reinforcement structure located on a first lateral side and a flexible, stress relief portion located on a second lateral side of said bendable portion opposite to said first lateral side,
c) a pull wire connected to said bendable portion and extending therefrom up to said proximal end portion along said lumen; and
d) an actuation region, being longitudinally elastically deformable, is adapted to impart a tension force on the pull wire resulting in a distally-directed force to the bendable portion and causing the bendable region to bend or curve away from a longitudinal axis of the steerable guidewire,
wherein
i) said pull wire is affixed to said proximal end portion and
ii) the actuation region is located along the elongated body, wherein
the elongated body and the bendable portion are formed by a solid tubular body and the bendable portion of the solid tubular body has a plurality of cutouts in the stress relief portion.

2. The steerable guidewire of claim 1, wherein the reinforcement structure is an integral part of the elongated body.

3. The steerable guidewire of claim 1, wherein the flexible, stress relief portion comprises the plurality of cut-outs positioned on one lateral side of the bendable portion.

4. A system comprising the steerable guidewire of claim 1 and an actuator adapted to impart a tension force on the pull wire, by acting on the actuation region, resulting in a distally-directed force to the bendable region.

5. The system of claim 4, wherein the actuator is a torque device comprising:
a) a first handle comprising:
an elongated body, a proximal end and a distal end, said handle defining a lumen along its entire length, and said distal end comprising means for gripping and ungripping the actuation region of the steerable guidewire;
b) a second handle comprising:
an elongated body, a proximal end and a distal end, said handle defining a lumen along its entire length, and said proximal end comprising means for gripping and ungripping the actuation region of the steerable guidewire;
wherein the first and second handles are arranged in a coaxial configuration so that they can perform a longitudinal relative displacement.

6. The system of claim 5, wherein said gripping and ungripping means comprise a spring collet clamp.

7. The system of claim 5, wherein at least one of said distal end of said first handle and said proximal end of said second handle comprises a tapered tip adapted to engage said gripping and ungripping means and/or tighten them upon a longitudinal relative displacement of said first and second handles.

8. The system of claim 5, wherein the first and second handles and are connected through an accordion-like, corrugated element.

9. The system of claim 5, wherein the first and second handles are connected through a spring clip.

10. The system of claim 5, wherein the first and second handles are arranged in a coaxial male-female configuration.

11. The system of claim 5 further comprises a first tapered fastener and a second tapered fastener adapted to releasably engage and tighten said gripping and ungripping means of said first and second handles, respectively.

12. The system of claim 10, wherein one of the first or second handle comprises a longitudinal slot along its body, and the other of the first or second handle comprises a protrusion adapted to fit and slide along said longitudinal slot, and the system comprises a gear rotational actuator adapted to engage said protrusion and operate upon rotation a longitudinal relative displacement of said first and second handles.

13. A system comprising the steerable guidewire of claim 1 and an actuator adapted to be arranged coaxially around the actuation region of the steerable guidewire and to impart a tension force on the pull wire, by acting on the actuation region, resulting in a distally-directed force to the bendable region, wherein the actuator is a torque device comprising:
a) a first handle comprising:
an elongated body, a proximal end and a distal end, said handle defining a lumen along its entire length, and said distal end comprising means for gripping and ungripping the actuation region of the steerable guidewire;
b) a second handle comprising:
an elongated body, a proximal end and a distal end, said handle defining a lumen along its entire length, and said proximal end comprising means for gripping and ungripping the actuation region of the steerable guidewire;
wherein the first and second handles are arranged in a coaxial configuration so that they can perform a longitudinal relative displacement.

14. The steerable guidewire of claim 1, wherein the reinforcement structure is made of metal comprising at least one of stainless titanium, steel, nickel titanium alloy, tungsten, cobalt, chrome, nickel, aluminium, copper, and/or molybdenum.

15. The steerable guidewire of claim 2, wherein the elongated body is made of the same material as the reinforcement structure.

16. The steerable guidewire of claim 1, wherein the cutouts have a triangular, rounded, or rectangular appearance.

17. The steerable guidewire of claim 1, wherein a ratio between an inner diameter of the steerable guidewire and an outer diameter of the steerable guidewire is between 1:10 and 1:1.1.

18. The steerable guidewire of claim 1, wherein the steerable guidewire has a diameter ranging from 0.007″ to 0.014″, 0.014″ to 0.018″, or 0.018″ to 0.038″.

19. The steerable guidewire of claim 1, wherein the distal end extends 0.5 cm to 1 cm, 1 cm to 5 cm or 5 cm to 10 cm.

20. The steerable guidewire of claim 1, wherein an exterior surface of the steerable guidewire is entirely or partially covered with an elastic, biocompatible coating or sheath.

21. The steerable guidewire of claim 1, wherein a cutout height is selected from a range of 5% to 95% of an outer diameter of the guidewire.

22. The steerable guidewire of claim 21, wherein a cutout height is selected from a range of 10% to 50% of an outer diameter of the guidewire.

23. The steerable guidewire of claim 16, wherein the cutouts having a triangular appearance have a cutout angle selected from a range of 5° to 165°.

24. The steerable guidewire of claim 23, wherein the cutout angle is selected from a range of 30° to 120°.

25. The steerable guidewire of claim 16, wherein a rounded cutout height of cutout having a rounded appearance is selected from a range of 5% to 95% of an outer diameter of the guidewire, with a longitudinal width of the cutout to a height of the cutout ratio between 0.01 and 10.

26. The steerable guidewire of claim 25, wherein the rounded cutout height is selected from a range of 50% to 75% of the outer diameter of the guidewire.

27. The steerable guidewire of claim 16, wherein the cutout having a rectangular appearance, has a cutout height selected from a range of 5% to 95% of an outer diameter of the guidewire.

28. The steerable guidewire of claim 27, wherein the cutout height is selected from a range of 50% to 75% of the outer diameter of the guidewire.

29. The steerable guidewire of claim 1, wherein the distal end portion has a lower stiffness or a similar stiffness as the elongated body.

30. A steerable guidewire having an elongated body defining a longitudinally-arranged lumen, said steerable guidewire comprising:
a) a proximal end portion;
b) a distal end portion comprising a bendable portion and a tip, said bendable portion comprising a reinforcement structure located on a first lateral side and a flexible, stress relief portion located on a second lateral side of said bendable portion opposite to said first lateral side,
c) a pull wire connected to said bendable portion and extending therefrom up to said proximal end portion along said lumen; and
d) an actuation region, being longitudinally elastically deformable, is adapted to impart a tension force on the pull wire resulting in a distally-directed force to the bendable portion and causing the bendable region to bend or curve away from a longitudinal axis of the steerable guidewire,
wherein
i) said pull wire is affixed to said proximal end portion and
ii) the actuation region is located along the elongated body, wherein
the elongated body and the bendable portion are formed by a solid tubular body and the bendable portion of the solid tubular body has a plurality of cutouts in the stress relief portion, wherein the elongated body is made of the same material as the reinforcement structure and the reinforcement structure is an integral part of the elongated body, and wherein the cutouts have a triangular, rounded, or rectangular appearance.
